SEQUENCE LISTING (1 sequence)

SEQ ID NO: 1 - protein, 13 aa, Streptococcus pyogenes
AHIVMVDAYKPTK

(19) United States
(12) Patent Application Publication
(10) Pub. No.: US 2021/0263024 A1
(43) Pub. Date: Aug. 26, 2021

(54) LANTHANIDE NANOP ARTICLE BASED FLUOROPHORES

(71) Applicants: ILISATECH, INC., Houston, TX (US); WILLIAM MARSH RICE UNIVERSITY, Houston, TX (US)

(72) Inventors: Xiaoyan Robert BAO, Houston, TX (US); Gang BAO, Houston, TX (US); Sheng Tong, Houston, TX (US)

(21) Appl. No.: 17/261,348

(22) PCT Filed: Jul. 19, 2019
(86) PCT No.: PCT/US19/42589

§ 371 Date: Jan. 19, 2021

Related U.S. Application Data
(60) Provisional application No. 62/701,734, filed on Jul. 21, 2018.

(51) Int. Cl. G01N 33/543; C09K 11/77; C07K 16/28; G01N 21/64

(52) U.S. Cl. CPC G01N 33/54346; G01N 21/6428; C07K 16/2836; C09K 11/7729

(57) ABSTRACT

A novel fluorophore based on an acid soluble reagent that is a lanthanide metal ion nanoparticle that is passivated and/or coated for water dispersion and to minimize nonspecific binding, and assays for same

This application claims priority to U.S. Ser. No. 62/701,734, filed Jul. 21, 2018, and incorporated by reference in its entirety for all purposes.

### FEDERALLY SPONSORED RESEARCH STATEMENT

Not applicable.

### REFERENCE TO MICROFICHE APPENDIX

Not applicable.

### FIELD OF THE DISCLOSURE

This invention relates to novel signal detector molecules that are longer lived than most background fluorescence and thus can be assayed by time resolved fluorescence. These can be employed in a variety of different assays types based on any ligand/target binding, and can be used in solid phase, bead based, or solution based assays.

### BACKGROUND OF THE DISCLOSURE

Enzyme-linked immunosorbent assays (ELISAs) are a standard workhorse technique in widespread use for the sensitive detection of biomolecules. A major advantage of ELISA is that it uses antibodies for detection. There are well-known techniques for raising high-affinity antibodies against almost any biomolecule of interest, and plenty of existing antibodies that enable ELISA-type techniques with little to no antibody development work.

However, ELISAs suffer from a series of disadvantages, including: (1) batch-to-batch variations in enzyme activity requiring the generation of a standard curve in every ELISA plate run; (2) signal instability necessitating extra quench steps after the proper development time, or the ability to read plates at defined time points, complicating its application in high throughput settings; and (3) both fluorescence and absorbance based ELISAs often require high-cost, high-sensitivity equipment for detection. These disadvantages are especially acute for deployment of ELISAs to field use in resource-poor regions.

Some of these disadvantages have been addressed by using single lanthanide ions or lanthanide nanoparticles incorporating lanthanide chelates to label detection antibodies¹. Herein, we describe an alternative to ELISAs that uses lanthanides to yield higher consistency and greater ease of detection, which promises to give even higher sensitivity than existing lanthanide-based techniques. It is called Lanthanide Nanoparticle-based Immunosorbent Assay (LANISA) and is based on our previously-published work using iron oxide nanoparticles².

### SUMMARY OF THE DISCLOSURE

At the core of LANISA is a lanthanide-containing metal oxide nanoparticle (FIG. 1) that is amenable to dissolution under acidic environments, resulting in the release of lanthanide ions. The metal oxide nanoparticles are coated to enable solubility and functionalization and prevent their nonspecific adsorption to surfaces. The nanoparticle surface passivation moieties can be modified to be cross-linkable to detection biomolecules. For instance, moieties can be maleimide modified so that they can be bound to immunoglobulin G (IgG)-based or immunoglobulin Y (IgY)-based antibodies whose inter-heavy chain disulfide bridges have been broken using a reducing agent. The nanoparticle surface can also be functionalized with streptavidin so the biotinylated IgG can be readily conjugated to the nanoparticles.

These passivated, coated nanoparticles can then be used to perform immunoassays (FIG. 2).

The invention includes any one or more of the following embodiment(s), in any combination(s) thereof:

A fluorogenic reagent comprising:

a) a lanthanide-containing metal oxide nanoparticle;

b) means for water dispersion of said lanthanide-containing metal oxide nanoparticle;

c) functionalization of said lanthanide-containing metal oxide nanoparticle with a linker or a ligand; and

d) said fluorogenic reagent being dissolvable at acid pH to release said lanthanide.

A fluorogenic reagent, said fluorogenic reagent comprising:

a) a passivated lanthanide-containing oxide nanoparticle that is water soluble;

b) said nanoparticle being covalently bound to a ligand;

c) said fluorogenic reagent being dissolvable at acidic pH to release said lanthanide.

A fluorogenic reagent, said fluorogenic reagent comprising:

a) a lanthanide-containing oxide nanoparticle coated with a polar lipid on an exterior surface thereof, said lanthanide being europium;

b) said polar lipid being covalently bound to a tag selected from the group consisting of an antibody, an antibody derivative, a biotin, a lectin, a streptavidin, a polyHIS tag, maleimide, a nucleic acid, a peptide, a protein, and a polysaccharide.

c) wherein said fluorogenic reagent is capable of dissolving at acid pH to release said lanthanide.

A fluorogenic reagent, said fluorogenic reagent comprising:

a) a lanthanide-containing oxide nanoparticle coated with DSPE-PEG on an exterior surface thereof, wherein said lanthanide is terbium or dysprosium or europium;

b) said exterior surface being functionalized with maleimide;

c) wherein said fluorogenic reagent is capable of dissolving at acid pH to release said lanthanide.

A fluorogenic reagent, said fluorogenic reagent comprising

a) a lanthanide-containing metal oxide nanoparticle;

b) a coating on said lanthanide-containing metal oxide nanoparticle to allow water solubility;

c) an optional functionalization of said coating to allow binding to a linker or a ligand; and

d) said fluorogenic reagent being dissolvable at acid pH (pH<7) to release said lanthanide.

Any fluorogenic reagent herein described, wherein said lanthanide is europium, terbium or dysprosium.

Any fluorogenic reagent herein described, wherein said metal oxide is terbium oxide or dysprosium oxide or europium oxide or lanthanide-doped iron oxide.

Any fluorogenic reagent herein described, wherein said coating is a polar lipid.

Any fluorogenic reagent herein described, wherein said coating is a functionalized polar lipid.

Any fluorogenic reagent herein described, wherein said coating is a polar lipid covalently bound to a tag selected from an antibody, an antibody derivative, a biotin, a lectin, a streptavidin, a polyHIS tag, a nucleic acid, a peptide, a protein, and a polysaccharide.

Any fluorogenic reagent herein described, wherein said coating is selected from DSPE-PEG, DSPE-PEG-NH2, DSPE-PEG-FA, DSPE-PEG-CHO, DSPE-PEG-NPC, DSPE-PEG-NHS, DSPE-PEG-MAL, DSPE-PEG-PDP, Bis-DSPE-PEG, DSPE-PEG-Cyanur, DSPE-PEG-Azide, DSPE-PEG Succinyl, DSPE-PEG-TMS, DSPE-PEG-Carboxylic Acid, DSPE-RGD, phosphatidyl choline, phosphatidyl ethanolamine, phophatidyl serine, phospholipids, and glucolipids.

Any fluorogenic reagent herein described, wherein the metal oxide is europium-doped iron oxide and the coating is DSPE-PEG or a functionalized derivative of DSPE-PEG.

A method of assaying a target; said method comprising:

a) contacting a sample suspected of containing a target with a surface capable of capturing said target;

b) contacting a solution containing any fluorogenic reagent herein described for a time sufficient for said reagent to directly or indirectly bind said target;

c) washing away unreacted fluorogenic reagent and assay components;

d) acidifying said sample to release said lanthanide;

e) chelating said lanthanide;

flash or continuously activating said sample for less than a second with a light of a wavelength absorbable by said lanthanide; and

measuring fluorescence of said lanthanide at least 1 millisecond after said flash or continuous activation.

A method of assaying a target; said method comprising:

a) contacting a solution containing a target with any fluorogenic reagent herein described for a time sufficient for said reagent to directly or indirectly bind said target;

b) washing away unreacted fluorogenic reagent and assay components;

c) acidifying said sample to release said lanthanide;

d) flash activating said sample for less than a second with a light of a wavelength absorbable by said lanthanide; and

e) measuring fluorescence of said lanthanide at least 1 millisecond after said flash activation.

Any method herein, wherein said fluorogenic reagent is on a solid substrate, or on an antibody which is on a solid substrate.

Any method herein, wherein said target is captured is on a solid substrate or an antibody or an antibody which is captured on a solid substrate. Antibodies can also be captured via yet another antibody. The solid substrate can be a multiwell microtiter plate, any container, a flat substrate such as glass or plastic slide with an array of spots thereon, a bead, a bead with an antibody thereon, a bead with magnetic element thereon, etc., etc.

Any method herein, wherein said method is a competitive assay; a non-competitive assay; a homogeneous immunoassay; a two-site, noncompetitive immunoassay; a competitive, heterogeneous immunoassay; a one-site, non-competitive immunoassay; a two-site, non-competitive immunoassay; or an array LANISA.

As used herein a “nanoparticle” is less than 1 micron in any one dimension, usually <500 nm, most preferred in the 5-25 or about 10 nm range. The nanoparticles described herein are generally spherical, but this is not essential.

As used herein, “flash” activation refers to a brief burst of the light at which the lanthanide will absorb energy, so that the decay fluorescence can be measured. The flash is <1 second, preferably less than a microsecond, and the signal is then measured over time or at least 1 millisecond later, preferably 0.2 seconds later. The times will of course vary depending on the context, what the background radiation sources are and which lanthanide was chosen for use.

As used herein, “passivation” prevents or minimizes non-specific binding to surfaces, such as the glass or plastic used in assay containers.

As used herein, the “coating” is a layer that surround the nanoparticle, and is preferably non-covalently bound thereto, but ionically or covalently bound coatings could also be used.

The coating is preferably a polar lipid. As used herein, a “polar lipid” will spontaneously form a bilayer in water, typically having a polar head and an apolar tail. Exemplary polar lipids include phosphatidyl choline (PC), phosphatidyl ethanolamine (PE), phophatidyl serine (PS), phospholipids, glucolipids.

Polar lipids alone may have difficulty forming the coating layer of nanoparticles, and thus may benefit from conjugating the nanoparticles with a molecule that assists in the coating, such as a hydrophobic residue, such as the oleyl used herein. The nanoparticle coating or “capping” molecules can be a mixture of oleic acid and oleylamine, oleic acid alone, or oleylamine alone, depending on the synthesis methods. Therefore, polar lipids can form a micellar coating via hydrophobic interactions with the capping molecules. Potentially, however, the polar lipids could form liposomes to encapsulate nanoparticles that are conjugated with hydrophilic capping molecules, such as citric acid, poly(acrylic acid) or polyamines.

A particularly preferred coating is DSPE-PEG or 1,2-Distearoyl-sn-glycero-3-phosphoethanolamine-poly(ethylene glycol) or any other lipid-PEG. Exemplary coatings include PLGA-PEG, PLA-PEG, PCL-PEG, lipid-PEGs, poly(L-lysine)-PEG, and poly(L-glutamic acid)-PEG, to name a few.

There are also a large number of functionalized derivatives of DSPE-PEG that could be used and conveniently are commercially available. These include, but are not limited to, DSPE-PEG-NH2 (1,2-distearoyl-sn-glycero-3-phosphoethanolamine-N-[amino(polyethylene glycol)), DSPE-PEG-CHO (1,2-distearoyl-sn-glycero-3-phosphoethanolamine), DSPE-PEG-NPC, DSPE-PEG-NHS (alpha-(1,2-distearoyl-sn-glycero-3-phosphoethanolamine)-omega-succinimidyl carbonate poly(ethylene glycol)), DSPE-PEG-MAL (1,2-distearoyl-sn-glycero-3-phosphoethanolamine-N-[maleimide(polyethylene glycol)), DSPE-PEG-PDP (1,2-distearoyl-sn-glycero-3-phosphoethanolamine-N-[PDP(polyethylene glycol)), Bis-DSPE-PEG Bis(1,2-distearoyl-sn-glycero-3-phosphoethanolamine)-N-[(polyethylene glycol), DSPE-PEG-Cyanur (1,2-distearoyl-sn-glycero-3-phosphoethanolamine-N-[cyanur(polyethylene glycol)), DSPE-PEG-Azide (“1,2-distearoyl-sn-glycero-3-phosphoethanolamine-N-[azido(polyethylene glycol)), DSPE-PEG Succinyl (1,2-distearoyl-sn-glycero-3-phosphoethanolamine-N-[succinyl(polyethylene glycol)), DSPE-PEG-TMS (1,2-distearoyl-sn-glycero-3-phosphoethanolamine-N-[10-(trimethoxysilyl)undecanamide(polyethylene glycol)), DSPE-PEG Carboxylic Acid (1,2-distearoyl-sn-glycero-3-phosphoethanolamine-N-[carboxy(polyethylene glycol)), and DSPE-RGD (1,2-distearoyl-sn-glycero-3-phosphoethanolamine-N44-(p-(cysarginylglycylaspartate-maleimidomethyl)cyclohexane-carboxamide]).

As used herein, the term “ligand” refers to a molecule that binds to a particular “target” molecule and forms a complex, usually a binary complex, but trinary complexes are also possible. The binding is preferably highly specific binding, however, in certain embodiments, the binding of an individual ligand to the target molecule can be with relatively low affinity and/or specificity. The “target” molecule is the molecule of interest (e.g., the target of the assay), and obviously, a molecule can be a ligand in one context, but a target in another.

Examples of ligand/target binding pairs include, but are not limited to, streptavidin/biotin; peptide/peptide such as spytag/spycatcher; nucleic acid/complementary nucleic acid; ligand/receptor; steroid/steroid receptor; antibody/antigen; enzyme/substrate; polyhistidine tag/metal ion (e.g., nickel, cobalt and copper); aptamer/target; zinc finger/DNA, and the like, and more are being discovered every day.

As used herein, “tag” refers to a member of a binding pair or binding triplet. It functions to provide a tag or handle, that the other member(s) of the binding pair/triplet can grab onto to form a complex.

A “lanthanide” is one of the 15 metallic chemical elements with atomic numbers 57 through 71 (see below). A preferred lanthanide is europium. The lanthanides are:

Lanthanum—atomic number 57 with symbol Ln

Cerium—atomic number 58 with symbol Ce

Praseodymium—atomic number 59 with symbol Pr

Neodymium—atomic number 60 with symbol Nd

Promethium—atomic number 61 with symbol Pm

Samarium—atomic number 62 with symbol Sm

Europium—atomic number 63 with symbol Eu

Gadolinium—atomic number 64 with symbol Gd

Terbium—atomic number 65 with symbol Tb

Dysprosium—atomic number 66 with symbol Dy

Holmium—atomic number 67 with symbol Ho

Erbium—atomic number 68 with symbol Er

Thulium—atomic number 69 with symbol Tm

Ytterbium—atomic number 70 with symbol Yb

Lutetium—atomic number 71 with symbol Lu

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| CHO | Aldehyde |
| DSPE | 1,2-Distearoyl-sn-glycero-3-phosphoethanolamine |
| ELISA | Enzyme Linked Immunosorbent Assay |
| Eu(acac)₃ | Europium(III) acetylacetonate (C₁₅H₂₁EuO₆) |
| FA | Fatty Acid |
| Fe(acac)₃ | iron(III) ace ylacetonate (C₁₅H₂₁FeO₆) |
| IgA | Immunoglobulin A |
| LANISA | Lanthanide Nanoparticle-based Immunosorbent |
| | Assay |
| NH2 | Amine |
| NHS | N-Hydroxysuccininnide |
| NPC | Nitrophenyl Carbonate |
| PDP | (2-pyridyldithio)propionate |
| PEG | Polyethylene glycol |
| PSA | Prostate specific antigen |
| RGD | Arginine-Glycine-Aspartic acid |
| SD | Standard deviation |
| SPDP | Succininnidyl 3-(2-pyridyldithio)propionate |
| SpyTag | A peptide from S. pyodenes (SPY) |
| | fibronectin-binding protein FbaB |
| | (AHIVMVDAYKPTK) |
| SpyCatcher | Binding partner peptide of SpyTag with high |
| | affinity |
| TCEP | Tris(2-carboxyethyl)phosphine |
| VCAM1 | Vascular Cell Adhesion Molecule 1 |

### BRIEF DESCRIPTION OF FIGURES

FIG. 1. Europium-containing metal oxide nanoparticle immunoassay reagent (not to scale). A europium-containing metal oxide nanoparticle is synthesized with oleyl chains on the surface (a 3:5 mixture of oleic acid and oleylamine that binds via ionic interactions), so that it may be coated with 1,2-distearoyl-sn-glycero-3-phosphoethanolamine-N-[amino(polyethylene glycol)-2000] (DSPE-PEG) an amphiphilic copolymer of polar lipid and PEG that renders it dispersible in water and passivates it. Binding antibodies can then be bound directly via DSPE-PEG molecules that are functionalized with maleimide.

FIG. 2. Assay design. Protocol is similar to that for ELISA, except that development involves dissolving the bound lanthanide-containing metal oxide nanoparticles followed by complexing of the released lanthanide ions with chelators and fluorescence enhancers.

FIG. 3. A. A TEM image of EuFeO nanoparticles. The size is 8.5±1.0 nm. The iron to europium molar ratio is 18.2. Scale bar=50 nm. Note that the variation in the darkness of the nanocrystals is caused by the difference in the alignment of the crystal lattice with respect to the electron beam. B. A representative Eu standard curve used to quantify the doping rate of Eu in EuFeO nanoparticles. Data represent mean±SD of three measurements. The doping rate of Eu in the nanoparticles shown in (A) was calculated as 5.2%.

FIG. 4. Detection sensitivity of europium-containing metal oxide nanocrystals. Europium-containing metal oxide nanocrystals were dissolved with hydrochloric acid and the resulting europium ions were chelated by mixing with DELFIA enhancement solution. The fluorescence intensity of the samples were measured using a microplate reader in time-resolved fluorescence mode with different settings. FIG. 4A. The integration time was set at 400 μs or 1000 μs. FIG. 4B. The receiver gain (the ratio of the output signal power to the input signal power of a receiver, usually expressed in decibels) was set at 150 or 200. FIG. 4C. Examination of fluorescence intensity generated by europium oxide nanocrystals at a concentration of 20 pg/mL.

FIG. 5. Modification of streptavidin for conjugation with EuFeO nanoparticles. In Step I, 3 mg of streptavidin was incubated with 20 mM of SPDP for 30 minutes at ambient temperature to generate pyridyldithiol-activated streptavidin. In Step II, pyridyldithiol-activated streptavidin was reduced by 50 mM of TCEP to generate sulfhydryl-activated streptavidin, which was subsequently conjugated with maleimide-functionalized EuₓFe_{y}O nanoparticles.

FIG. 6. LANISA sandwich assay and detection sensitivity. An assay for detecting human IgA has a format similar to a typical ELISA. A. The detection antibody (mouse anti-human IgA antibody) is coated onto the microplate. After the solution containing human dimeric IgA was incubated in the plate, the bound human IgA is detected with a biotinylated rabbit anti-human IgA antibody. The EuFeO nanoparticles conjugated with streptavidin are then added and bind to the biotins on the rabbit antibodies. After unbound EuFeO nanoparticles are removed from the plate, the nanoparticles are dissolved and the released Eu ions are reacted with a fluorogenic chelator to generate fluorophores, which are quantified using a fluorescence plate reader. B. Fluorescence measurements show high sensitivity and a linear correlation between signal and IgA concentration over a wide range of concentrations.

FIG. 7. High sensitivity and wide dynamic range of LANISA in quantifying human PSA. PSA was chosen as a model biomarker to demonstrate the broad linear range of LANISA. A serial dilution of purified human PSA was quantified with LANISA, showing the high sensitivity (Limit of Detection=3.7 pg/mL) and wide linear range (four orders of magnitude, from 30 to 0.0037 ng/mL, y=703.28x, R²=0.9925) of LANISA. Data represent mean±SD of three measurements.

FIG. 8. Quantification of pancreatic cancer marker Carbohydrate antigen 19-9 (CA19-9). A. Standard curve for CA19-9 quantification. Serum samples were created by adding known amount of purified human CA19-9 to pooled normal persons' serum at designated concentrations. The fluorescence signals of these samples were then quantified with a sandwich LANISA assay. B. Quantification of CA19-9 using clinical samples. Sera from 24 pancreatic cancer patients were thawed to room temperature, diluted 1 to 5 with BSA buffer, and the concentration on CA19-9 was quantified with a sandwich LANISA assay similar to that shown in FIG. 6. The concentration of CA19-9 in pooled serum from healthy persons was measured as the control.

FIG. 9. Bead-based assay design. Analyte is captured on antibody-coated magnetic beads to improve assay performance.

FIG. 10. Array-LANISA. LANISA can be combined with a microarray format to have multiplexed detection of analytes. The microarray can be formed by wells in a high-binding microplate, or by an array of spots on a paper, glass, plastic, or other solid surfaces. Each well or spot in a microarray is filled with capture molecules for the detection of a specific analyte, and fluorescence signal in each well or spot will be generated and quantified individually. The array LANISA can be used for multiplexed detection of proteins, carbohydrate groups, nucleic acids and their combinations.

### DETAILED DESCRIPTION

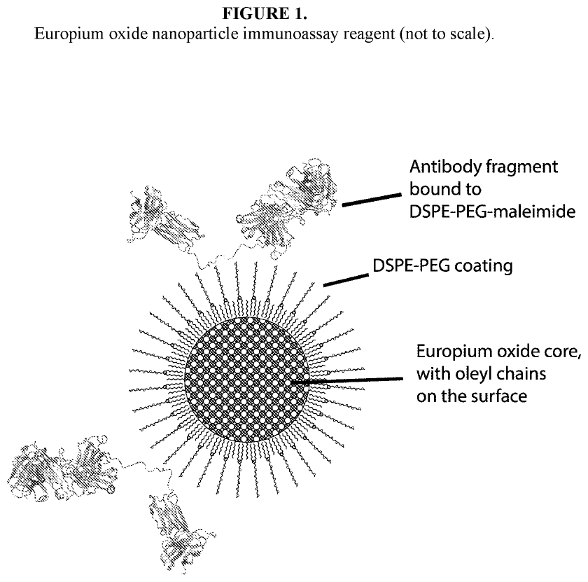

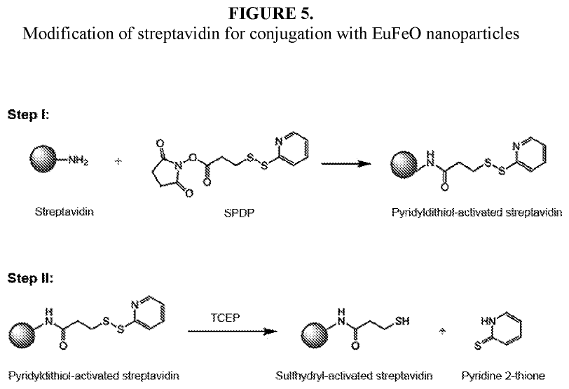

As an example, a full protocol for detection of Vascular Cell Adhesion Molecule 1 (VCAM1) using the scheme shown in FIG. 1 is as follows. Alternatively, the following steps can be modified to use biotinylated binding antibodies that can be bound to lanthanide-containing metal oxide nanoparticles functionalized with streptavidin (FIG. 5).

### Step I. Nanoparticle Synthesis

This protocol was modified from the published literature³. Europium oxide (Eu₂O₃) powder was dissolved to 75 mM in deionized water with HNO₃. It is then complexed to 1-benzoylacetone (HBA) by mixing in two volumes of 0.3 M HBA, and precipitated by dropwise addition of ammonia. The precipitate was collected by filtration, washed with water, and dried under vacuum. 0.2 mmol of the precipitate was then added to 15 ml of a 3:5 mixture of oleic acid and oleylamine. The solution was bubbled with argon, incubated under vacuum at 100° C. for 1 hr to remove solvents, and heated to 310° C. under argon at 20 K/min. It was then held at this temperature under argon for 1 hr. After the reaction, an excess of ethanol was added and the nanoparticles isolated by centrifugation. Nanoparticles were then washed with ethanol and dispersed in toluene. We note that similar synthesis protocols can be used for samarium, gadolinium, and terbium oxides³, the lanthanide ions of which all exhibit strong fluorescence in aqueous solution⁴.

Alternatively, nanoparticles may be mostly iron oxide and doped with lanthanides. This protocol was developed according to a published method⁵. Lanthanide-dope iron oxide nanoparticles with uniform size distribution were synthesized in two steps: seed synthesis and seed-mediated growth. The first step was to synthesize nanocrystals of 5-6 nm in diameter as the seeds. 5 mmol Fe(acac)₃, 1 mmol Eu(acac)₃, 24 mmol oleic acid, 24 mL benzyl ether and 12 mL of squalane were mixed in a 100 mL flask. The mixture was heated to 200° C. at a ramping rate of 4° C. per minute and incubated at this temperature for 2 hours under an argon flow. Then the mixture was heated to reflux at a ramping rate of 4° C. per minute and incubated for 1 hour under an argon flow. After reaction, the mixture was cooled down to room temperature and the nanocrystals collected by precipitation with acetone.

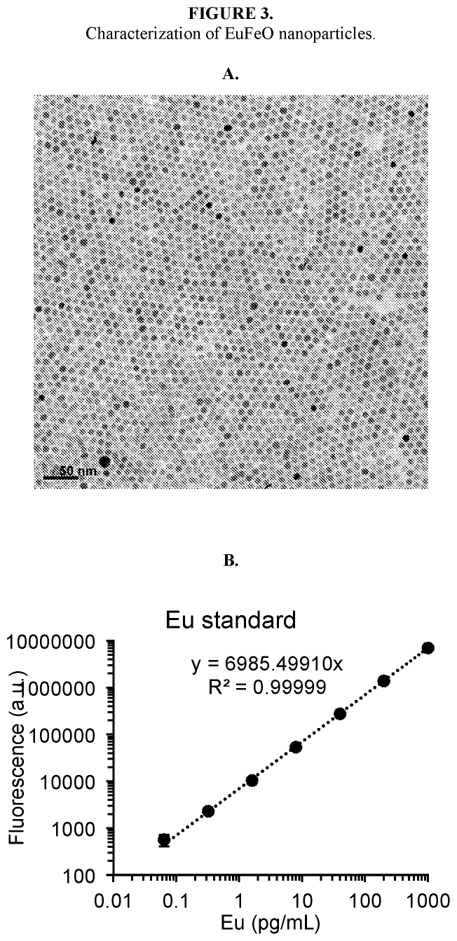

In a typical seed mediated growth step, 86 mg Fe of seeds dispersed in 10 mL of toluene, 2.5 mmol Fe(acac)₃, 0.5 mmol Eu(acac)₃, 24 mmol oleic acid, 24 mL benzyl ether and 12 mL of squalane are mixed in a 100 mL flask. The mixture was heated to 200° C. for 2 hours and to 300° C. for 30 minutes. The resulting nanocrystals were collected by precipitation with acetone. A TEM image of EuFeO nanoparticles is shown in FIG. 3.

### Step II. Passivation Coating

The protocol for this and subsequent steps was adapted from previously published work. 0.4 ml of a Eu₂O₃ nanocrystal suspension (15 mg Eu/ml in toluene) were mixed with 2 ml chloroform containing 12 mg DSPE-mPEG (the PEG chain ends with a methoxy group) and 0.24 mg DSPE-PEG-maleimide in a 250 ml flask 10 ml DMSO added to the flask dropwise with gentle shaking over 20 min.

Chloroform and toluene were removed by putting the suspension under vacuum until 8.8 g suspension remains. 16 ml of deionized water was then slowly added to the mixture, and the DMSO removed by filtration through a Vivaspin centrifugal filter tube (m.w. cutoff=100 kD). Coated nanoparticles were collected by two centrifugations at 50000×g, 4° C. for 1 hr, and resuspended in deionized water.

### Step III. Conjugation of Detection Antibodies

100 μg goat anti-human VCAM1 antibodies (IgG) were reduced by mixing with 200 μl PBS-EDTA containing 100 mM 2-mercaptoethylamine-HCl and incubating at 37° C. for 2 hr. The antibody fragments were washed 6× with sodium acetate (100 mM, pH 5.5) in Amicon centrifugal filters (m.w. cutoff=10 kD). Reduced antibody fragments are then mixed with coated Eu₂O₃ nanoparticles (3 antibody fragments per coated nanoparticle), and phosphate buffered saline (PBS) added to adjust the pH of the solution to 7.2. This mixture was incubated at room temperature overnight. After incubation, nanoparticles were collected by two centrifugations at 50000×g, 4° C. for 1 hr, and the supernatant removed. Conjugated nanoparticles were then resuspended at 300 μg Eu/ml in deionized water.

### Step IV. Immunoassay

To generate capture surfaces for VCAM1 immunoassays, wells in high-binding microplates (e.g. Nunc™ Maxisorp™ 96 plates) were filled with 100 μl of capture antibody solution (mouse anti-VCAM1, 10 μg/ml) and incubated for 24 hrs at 4° C. They were then washed 3× with PBS+0.05% tween-20 and incubated for another 24 hrs at 4° C. with 300 μl 1% BSA in PBS, after which they were ready to use. To capture VCAM1 molecules, 100 μl VCAM1-containing cell lysate (0.8-3 μg/ml protein) was incubated in wells at 37° C. with shaking for 1 hr. The lysate was then removed, and the well washed 3× with PBS+0.05% Tween-20. Conjugated nanoparticle suspension from step III was then added (100 μl), incubated at 37° C. with shaking for 1 hr, and unbound suspension removed. The well was washed 3× with PBS+0.05% tween-20.

### Step V. Development and Quantitation

Bound nanoparticles were dissolved by adding 50 μl 6 M HCl to the well and incubated at room temperature for 15 min. Then 35 μl 8 M NaOH and 50 μl of 4 M ammonium acetate solution were added sequentially to neutralize the solution. Luminescence was then developed by adding 50 μl DELFIA enhancement solution (Perkin Elmer).

The DELFIA® Enhancement Solution is an acidic chelating detergent solution intended for use in the quantitative determination of Eu3+/Sm3+ when using a time-resolved fluoroimmunoassay. The solution dissociates Eu3+/Sm3+ from solid phase bound Eu-labeled antibodies or proteins during a time period of a few minutes to form a homogeneous and highly fluorescent micellar chelate solution. The solution allows highly sensitive Eu3+/Sm3+ measurements to be made when using the time-resolved fluorometer. The DELFIA® Enhancement Solution is also used as part of Tb3+/Dy3+ measurement together with DELFIA® Enhancer.

The well can then be read in a standard plate reader in time-resolved fluorescence mode, with excitation at 320 nm, 400 μs delay, and 400 μs integration time.

### Preliminary Results

The use of lanthanide nanoparticles can potentially achieve large signal gains: a 40 nm diameter pure Eu₂O₃ nanoparticle is estimated to contain approximately 1.0 million europium atoms, thereby yielding 1 million fluorophores from a single binding event. As far as we are aware, this level of amplification gain would be one of the highest reported for an immunosorbent assay.

The fluorophores so generated have the additional advantage that their fluorescence is long-lived. Therefore, they can be quantitated using time-resolved fluorescence. In this mode, fluorophores in the sample are excited by a burst of light but quantitated a short time (>100 μs) after the end of that burst. Since most contaminating fluorescence has much shorter lifetimes (ns), these will all have decayed by the time signal integration starts, so that background autofluorescence will not affect measurements. This reduces the noise floor of quantitation and increases detection sensitivity. Furthermore, because excitation light is not present during quantitation, no optical filters are needed in the quantitation device⁶ as long as the detection apparatus can be made sufficiently light-tight. This significantly reduces instrument cost and complexity.

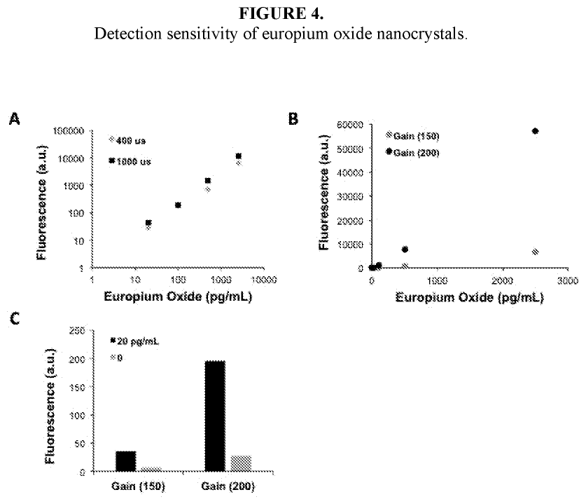

In a preliminary experiment (FIG. 4), we used commercially available europium nanoparticles. Europium oxide nanocrystals (Sigma-Aldrich) were dissolved with hydrochloric acid. The solution was diluted serially to designed concentrations. After that, the solutions were mixed with DELFIA enhancement solution (Perkin Elmer) at 1:1 ratio in a corning 384-well plate. The fluorescence signal from europium chelates was measured at 615 nm with excitation at 320 nm and 400 us delay.

There is a good linearity when the concentration of europium oxide increases from 20 pg/mL to 2500 pg/mL (FIGS. 4A and 4B) under different detection conditions (the integration time equals 400 μs or 1000 μs and the gain equals 150 or 200 respectively). FIG. 4C shows that the fluorescence signal can be readily detected at a concentration as low as 20 pg/mL, with a noise floor of ˜3 pg/ml (which can be further reduced).

To functionalize EuFeO nanoparticles, we modified streptavidin as shown in FIG. 5. Specifically, 3 mg of streptavidin was incubated with 20 mM of SPDP for 30 minutes at ambient temperature to generate pyridyldithiol-activated streptavidin (Step I). Pyridyldithiol-activated streptavidin was then reduced by 50 mM of TCEP to generate sulfhydryl-activated streptavidin (Step II). The modified streptavidin was subsequently conjugated to maleimide-modified EuFeO nanoparticles.

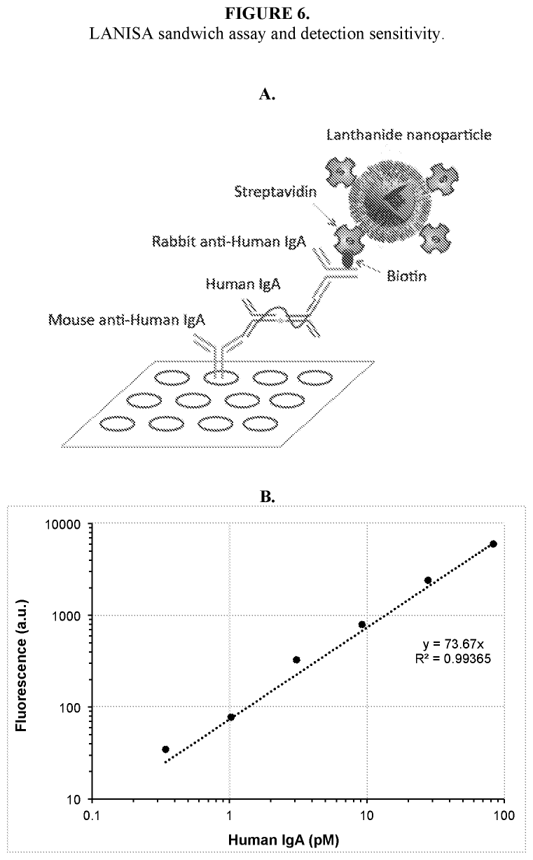

In a subsequent experiment, we synthesized europium-doped iron oxide (EuFeO) nanoparticles and coated them with DSPE-PEG. These nanoparticles were then functionalized with streptavidin (FIG. 6A). To quantify the concentration of human IgA in a solution, we first coated a microplate with mouse anti-human IgA antibody. For the detection, 50 μL of the solutions containing human IgA was added to each well in the microplate and incubated on a shaker for 1 hour at ambient temperature.

After that, the plate was washed with PBS with 0.05% Tween-20. Each well was then incubated with a biotinylated rabbit anti-human IgA antibody (50 μL, 2 μg/mL) for 1 hr. After washes, the plate was incubated with the streptavidin-functionalized EuFeO nanoparticles (50 μL, 50 μg/mL) for 1 hr. After unbound EuFeO nanoparticles were removed by additional washes, the fluorescence signal was developed and detected as mentioned above. As shown in FIG. 6B, our measurements demonstrate a high detection sensitivity and a linear correlation between fluorescence signal and IgA concentration over a wide range of concentrations.

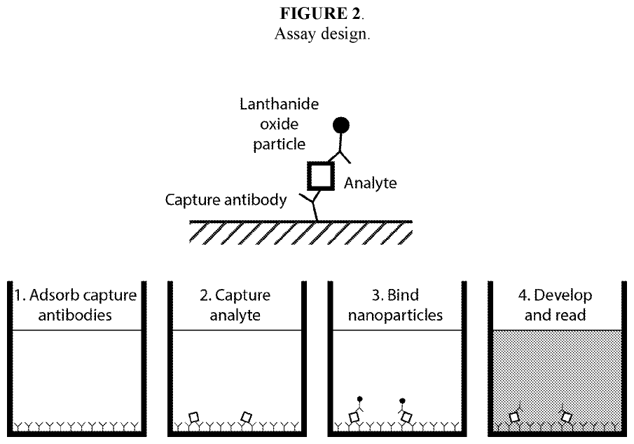

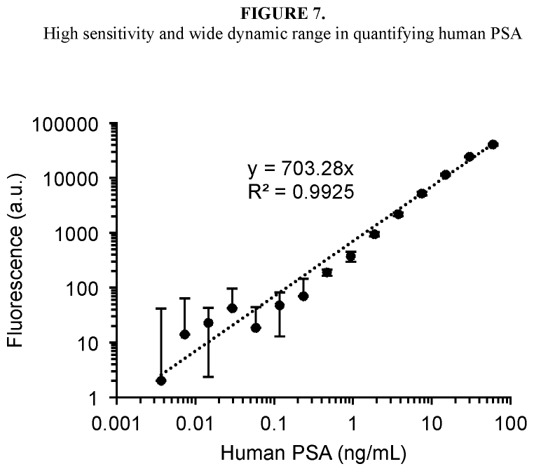

We also demonstrated the high sensitivity and wide dynamic range of LANISA in quantifying human PSA (FIG. 7). A serial dilution of purified human PSA was performed and the concentrations of PSA were quantified using the scheme shown in FIG. 2. We achieved high sensitivity (Limit of Detection=3.7 pg/mL) and a very wide dynamic range (from 30 to 0.0037 ng/mL), as well as a linear correlation between fluorescence signal and PSA concentration over four orders of magnitude.

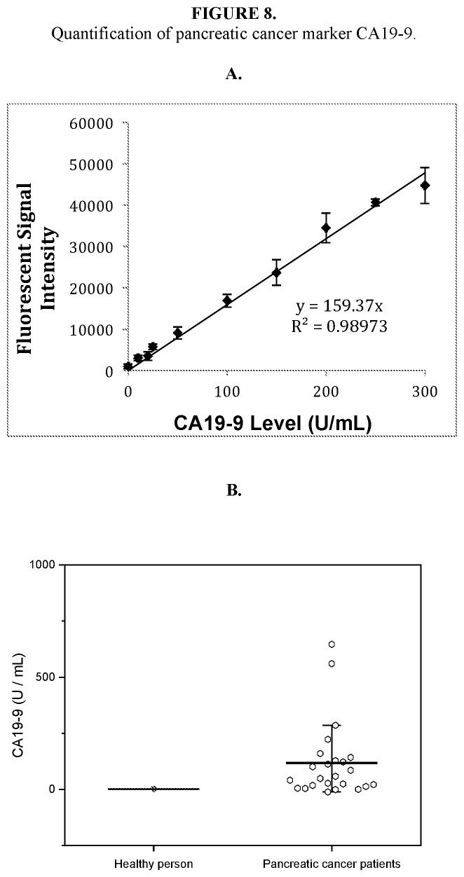

We further quantify the pancreatic cancer marker CA19-9 in the sera of pancreatic cancer patients using a sandwich LANISA assay. We first generated a standard curve by measuring fluorescence signals of serum samples with known concentrations of purified human CA19-9 (FIG. 8A). We then measured the fluorescence signals in serum samples from 24 pancreatic cancer patients using a sandwich LANISA assay, and determined the CA19-9 concentrations (in U/ml) (FIG. 8B) using the standard curve in FIG. 8A. Compared with CA19-9 concentration of healthy persons, most of the pancreatic cancer patients showed a elevated level of CA19-9, consistent with what reported in the literature⁷.

### Variations

Lanthanide nanoparticles. Many variants can be made to the above protocol. Several different synthetic routes to Eu₂O₃ nanoparticles have been reported⁸⁻¹⁰, and other studies have used commercially purchased nanoparticles¹¹. In addition to europium oxide nanoparticles, other lanthanide nanoparticles may be used, including terbium oxide and dysprosium oxide nanoparticles. The use of different lanthanide nanoparticles in LANISA may enable multiplexing.

Coating moieties. Further, there are several commercially available DSPE-PEG conjugates of interest here, including those with amine, carboxy, biotin, azide, and DBCO groups (Avanti Polar Lipids, Inc.). These groups allow crosslinking via NHS esters, carbodiimide, streptavidin, copper-catalyzed click chemistry, and copper-free click chemistry, respectively.

Detection molecules. In addition to biomolecule detection with antibodies, other capture systems include lectins that bind to carbohydrate groups with specificities that match those of antibody-antigen interactions¹² and use of nucleic acids that can be designed to specifically hybridize to sequences of interest with tunable specificity and sensitivity¹³. Nanoparticle functionalization with lectins can be accomplished using NHS or carbodiimide¹⁴, and functionalization with nucleic acids can be accomplished with any of the above-mentioned techniques by using oligonucleotides synthesized with the relevant functionalization groups.

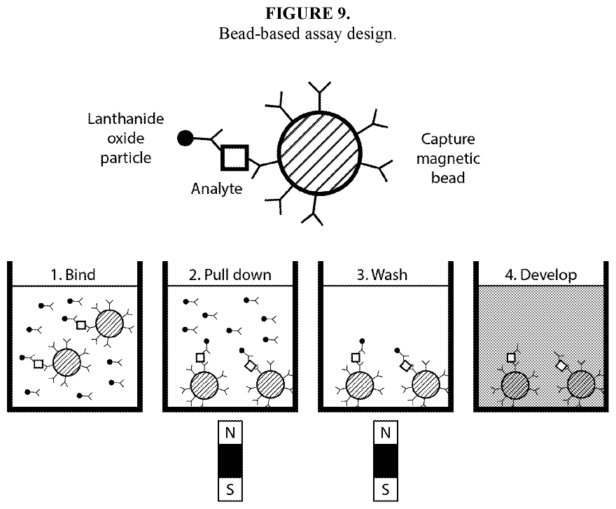

Solution phase assays. One major drawback of surface-based immune assays is that analyte binding requires extended incubation periods. While this is less of a problem in laboratory experiments, use of these assays in clinical settings can benefit from having shorter assay times. We propose to overcome this drawback by using solution phase analyte capture with magnetic beads. As shown in FIG. 9, capture antibodies can be attached to magnetic beads rather than well bottoms, thereby allowing beads to interact with the biological sample. This scheme greatly increases the amount of surface area available for capture antibody conjugation. Even without an increased amount of surface area, the ability to distribute that surface area throughout the biological sample volume will greatly speed up binding.

A magnetic bead-based immunoassay protocol would be as follows:

Conjugate 100 μg mouse anti-VCAM1 antibodies to 100 μl ProMag® Bind-IT™ pre-activated magnetic microspheres (Bangs Labs) according to manufacturer's recommendation (protocol attached). Wash 10 μl antibody-conjugated microspheres with PBS+1% BSA. Resuspend to 10 μl in PBS+1% BSA and mix with 100 μl VCAM-1-containing cell lysate in a plain polystyrene 96-well plate. Incubate with gentle shaking at 37° C., 15 min. Add 50 μl suspension of antibody-conjugated lanthanide oxide nanoparticles and incubate with gentle shaking at 37° C. for another 15 min. Chill the plate to 4° C. and bind magnetic beads to the well bottom using a rare earth magnet. Remove supernatant and resuspend with 200 μl ice-cold PBS+1% BSA. Repeat binding, supernatant removal, and resuspension three times. Bind beads, remove supernatant, and resuspend using 50 μl 6 M HCl. Incubate at room temperature with gentle shaking, 15 min. Mix in 50 μl DELFIA enhancement solution and read on plate reader, as described previously.

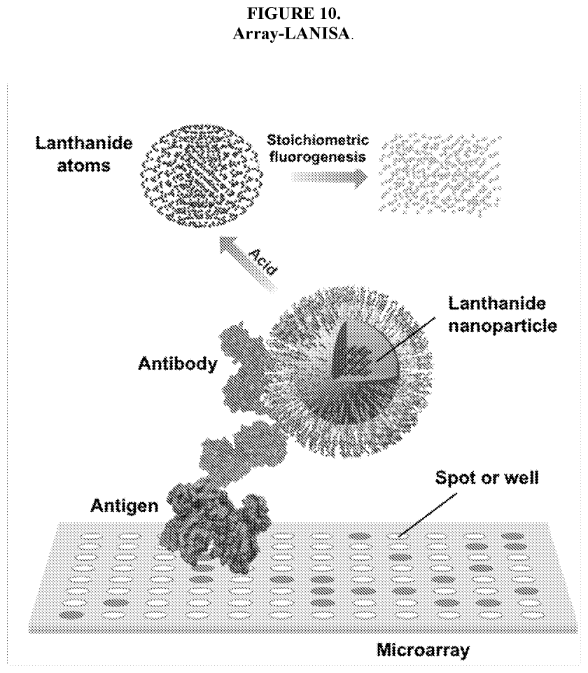

Array LANISA. LANISA can be generalized to a microarray format to have multiplexed detection of analytes (FIG. 10). In one embodiment, wells in a high-binding microplate will be filled with different capture molecules specific to the analytes (e.g., proteins), and the lanthanide nanoparticles will be functionalized to target different analytes. A biological sample with different analytes will be incubated with the microplate and, following the steps I to IV described above to remove the unbound analytes and nanoparticles, fluorescence signal in each well will be generated and quantified individually using a procedure similar to that described in Step V above.

In another embodiment, a microarray of spots printed on paper will be used instead of a microplate. The capture molecules corresponding to a specific analyte will be immobilized at the spot through the thickness of the paper, together with lanthanide chelators, such as Tris chelates, tetrakis chelates, phthalates, picrate, diethylenetriaminepentaacetic acid (DTPA), triethylenetetraminehexaacetic acid (TTHA), 2,2′,2″,2′″-[[4′-(aminobiphenyl-4-yl)-2,2′:6′,2″-terpyridine-6,6″-diyl]bis(methylenenitrilo)]tetrakis(acetato) (ATBTA) and its derivatives, 1,4,7,10-tetraazacyclododecane-1,4,7,10-tetrakis(acetamidoacetic acid) (DOTA), and salicylate complexes. Acid (such as hydrochloric acid, nitric acid, acetic acid, and sulfuric acid) will be applied to the entire microarray for signal development and the fluorescence signal at each spot will be quantified. In this embodiment, chelators would be immobilized on paper fibers and be concentrated enough to suppress diffusion of lanthanide ions released by the dissolution of the oxide nanoparticles. The array LANISA can be used for multiplexed detection of proteins, carbohydrate groups, nucleic acids and their combinations.

Competitive, homogeneous immunoassays. In a competitive, homogeneous immunoassay, unlabeled analyte in a sample competes with labeled analyte to bind an antibody. The amount of labeled, unbound analyte is then measured. In theory, the more analyte in the sample, the more labeled analyte gets displaced and then measured; hence, the amount of labeled, unbound analyte is proportional to the amount of analyte in the sample.

Two-site, noncompetitive immunoassays. These usually consist of an analyte “sandwiched” between two antibodies. ELISAs are often run in this format.

Competitive, heterogeneous immunoassays. As in a competitive, heterogeneous immunoassay, unlabeled analyte in a sample competes with labeled analyte to bind an antibody. In the heterogeneous assays, the labeled, unbound analyte is separated or washed away, and the remaining labeled, bound analyte is measured.

One-site, noncompetitive immunoassays. The unknown analyte in the sample binds with labeled antibodies. The unbound, labeled antibodies are washed away, and the bound, labeled antibodies are measured. The intensity of the signal is directly proportional to the amount of unknown analyte.

Two-site, noncompetitive immunoassays. The analyte in the unknown sample is bound to the antibody site, then the labeled antibody is bound to the analyte. The amount of labeled antibody on the site is then measured. It will be directly proportional to the concentration of the analyte because the labeled antibody will not bind if the analyte is not present in the unknown sample. This type of immunoassay is also known as a sandwich assay as the analyte is “sandwiched” between two antibodies.

The following reference are incorporated by reference in its entirety for all purposes.
1. Hagan, A. K. & Zuchner, T. Lanthanide-based time-resolved luminescence immunoassays. Anal Bioanal Chem 400, 2847-2864 (2011).
2. Tong, S., Ren, B., Zheng, Z., Shen, H. & Bao, G. Tiny grains give huge gains: nanocrystal-based signal amplification for biomolecule detection. ACS Nano 7, 5142-5150 (2013).
3. Si, R., Zhang, Y.-W., You, L.-P. & Yan, C.-H. Rare-earth oxide nanopolyhedra, nanoplates, and nanodisks. Angew. Chem. Int. Ed. Engl. 44, 3256-3260 (2005).
4. Elbanowski, M., Lis, S. & Konarski, J. Fluorescence of lanthanide (III) complexes with aminopolyacetic acids in aqueous solutions. Monatshefte für Chemie 118, 907-921 (1987).
5. De Silva, C. R. et al. Lanthanide(III)-doped magnetite nanoparticles. J Am Chem Soc 131, 6336-6337 (2009).
6. Connally, R., Jin, D. & Piper, J. High intensity solid-state UV source for time-gated luminescence microscopy. Cytometry A 69, 1020-1027 (2006).
7. Xing, H. et al. Diagnostic Value of CA 19-9 and Carcinoembryonic Antigen for Pancreatic Cancer: A Meta-Analysis. Gastroenterol Res Pract 2018, U.S. Pat. No. 8,704,751 (2018).
8. Wakefield, G., Keron, H., Dobson, P. & Hutchison, J. Synthesis and Properties of Sub-50-nm Europium Oxide Nanoparticles. J Colloid Interface Sci 215, 179-182 (1999).
9. Nguyen, T.-D. & Do, T.-O. General Two-Phase Routes to Synthesize Colloidal Metal Oxide Nanocrystals: Simple Synthesis and Ordered Self-Assembly Structures. J. Phys. Chem. C 113, 11204-11214 (2009).
10. Bazzi, R. et al. Synthesis and properties of europium-based phosphors on the nanometer scale: Eu2O3, Gd2O3:Eu, and Y2O3:Eu. J Colloid Interface Sci 273, 191-197 (2004).
11. Feng, J. et al. Functionalized Europium Oxide Nanoparticles Used as a Fluorescent Label in an Immunoassay for Atrazine. Anal. Chem. 75, 5282-5286 (2003).
12. Ghazarian, H., Idoni, B. & Oppenheimer, S. B. A glycobiology review: carbohydrates, lectins and implications in cancer therapeutics. Acta Histochem. 113, 236-247 (2011).
13. Wu, L. R. et al. Continuously tunable nucleic acid hybridization probes. Nat. Methods 12, 1191-1196 (2015).
14. Ezpeleta, I. Preparation of lectin-vicilin nanoparticle conjugates using the carbodiimide coupling technique. International Journal of Pharmaceutics 142, 227-233 (1996).

1. A fluorogenic reagent, said fluorogenic reagent comprising
a) a lanthanide-containing metal oxide nanoparticle;
b) a coating on said lanthanide-containing metal oxide nanoparticle to allow water solubility;
c) an optional functionalization of said coating to allow binding to a linker or a ligand; and
d) said fluorogenic reagent being dissolvable at acid pH to release said lanthanide.

2. The fluorogenic reagent of claim 1, wherein said lanthanide is europium, terbium or dysprosium.

3. The fluorogenic reagent of claim 1, wherein said metal oxide is terbium oxide or dysprosium oxide or europium oxide or lanthanide-doped iron oxide.

4. The fluorogenic reagent of claim 1, wherein said coating is a polar lipid.

5. The fluorogenic reagent of claim 1, wherein said coating is a functionalized polar lipid.

6. The fluorogenic reagent of claim 1, wherein said coating is a polar lipid covalently bound to a tag selected from an antibody, an antibody derivative, an antigen, a biotin, a lectin, a streptavidin, a polyHIS tag, a nucleic acid, a peptide, a protein, and a polysaccharide.

7. The fluorogenic reagent of claim 1, wherein said coating is selected from DSPE-PEG, DSPE-PEG-NH2, DSPE-PEG-FA, DSPE-PEG-CHO, DSPE-PEG-NPC, DSPE-PEG-NHS, DSPE-PEG-MAL, DSPE-PEG PDP, Bis-DSPE PEG, DSPE-PEG Cyanur, DSPE-PEG Azide, DSPE-PEG Succinyl, DSPE-PEG-TMS, DSPE-PEG Carboxylic Acid, DSPE-RGD, phosphatidyl choline, phosphatidyl ethanolamine, phophatidyl serine, phospholipids, and glucolipids.

8. The fluorogenic reagent of claim 1, wherein the metal oxide is europium-doped iron oxide and the coating is DSPE-PEG or a functionalized derivative of DSPE-PEG.

9. A fluorogenic reagent, said fluorogenic reagent comprising:
a) a lanthanide-containing metal oxide nanoparticle;
b) means for water dispersion of said lanthanide-containing metal oxide nanoparticle;
c) functionalization of said lanthanide-containing metal oxide nanoparticle with a linker or a ligand; and
d) said fluorogenic reagent being dissolvable at acid pH to release said lanthanide.

10. A fluorogenic reagent, said fluorogenic reagent comprising:
a) a passivated lanthanide-containing oxide nanoparticle that is water soluble;
b) said nanoparticle being covalently bound to a ligand;
c) said fluorogenic reagent being dissolvable at acidic pH to release said lanthanide.

11. A fluorogenic reagent, said fluorogenic reagent comprising:
a) a lanthanide-containing oxide nanoparticle coated with a polar lipid on an exterior surface thereof, said lanthanide being europium;
b) said polar lipid being covalently bound to a tag selected from an antibody, an antibody derivative, a biotin, a lectin, a streptavidin, a polyHIS tag, maleimide, a nucleic acid, a peptide, a protein, and a polysaccharide.
c) wherein said fluorogenic reagent is capable of dissolving at acid pH to release said lanthanide.

12. A fluorogenic reagent, said fluorogenic reagent comprising:
a) a lanthanide-containing oxide nanoparticle coated with DSPE-PEG on an exterior surface thereof, wherein said lanthanide is terbium or dysprosium or europium;
b) said exterior surface being functionalized with maleimide;
c) wherein said fluorogenic reagent is capable of dissolving at acid pH to release said lanthanide.

13. A method of assaying a target; said method comprising:
a) contacting a sample suspected of containing a target with a surface capable of capturing said target;
b) contacting a solution containing the fluorogenic reagent of claim 1 for a time sufficient for said reagent to directly or indirectly bind said target;
c) washing away unreacted fluorogenic reagent and assay components;
d) acidifying said sample to release said lanthanide;
e) chelating said lanthanide;
f) flash or continuously activating said sample for less than a second with a light of a wavelength absorbable by said lanthanide; and
g) measuring fluorescence of said lanthanide at least 1 millisecond after said flash or continuous activation.

14. A method of assaying a target; said method comprising:
a) contacting a solution containing a target with the fluorogenic reagent of claim 1 for a time sufficient for said reagent to directly or indirectly bind said target;
b) washing away unreacted fluorogenic reagent and assay components;
c) acidifying said sample to release said lanthanide;
d) chelating said lanthanide;
e) activating said sample with a light of a wavelength absorbable by said chelated lanthanide; and
f) measuring fluorescence of said chelated lanthanide.

15. The method of claim 14, wherein said fluorogenic reagent binds to the target captured on a solid substrate.

16. The method of claim 14, wherein said fluorogenic reagent binds to the target captured on an antibody on a solid substrate.

17. The method of claim 14, wherein said target is captured on a solid substrate.

18. The method of claim 14, wherein said target is captured on an antibody.

19. The method of claim 14, wherein said target is captured on an antibody on a solid substrate.

20. The method of claim 14, wherein said method is a competitive assay; a non competitive assay; a homogeneous immunoassay; a two-site, noncompetitive immunoassay; a competitive, heterogeneous immunoassay; a one-site, noncompetitive immunoassay; a two-site, noncompetitive immunoassay; or an array LANISA.

21. The method of claim 14, wherein step d and e include flash activating said sample for less than a second with a light of a wavelength absorbable by said lanthanide; and measuring fluorescence of said lanthanide at least 1 millisecond after said flash activation.
